(19) United States
(12) Patent Application Publication
(10) Pub. No.: US 2013/0324958 A1
(43) Pub. Date: Dec. 5, 2013

(54) Highly Flexible Absorbent Article Having Stiffened Landing Zone

(71) Applicants: Arman Ashraf, Mason, OH (US); Fang Liu, Mason, OH (US)

(72) Inventors: Arman Ashraf, Mason, OH (US); Fang Liu, Mason, OH (US)

(21) Appl. No.: 13/484,820

(22) Filed: May 31, 2012

(51) Int. Cl. A61F 13/51; A61F 13/62

(52) U.S. Cl. 604/367; 604/391

(57) ABSTRACT

A disposable diaper of a relatively thin design is disclosed. The diaper may have a maximum caliper of 3 mm and a minimum circular bending stiffness of 3 N within a Front Hip flex Zone. Various materials and structures that may be included to impart circular bending stiffness within a Front Hip Flex Zone are disclosed. While the relatively thin design may provide for wearer comfort and economy in packaging and shipping, the imparted stiffness may help improve fit characteristics and fastener attachment integrity

### BACKGROUND OF THE INVENTION

The business of manufacturing and selling disposable child diapers is highly competitive and capital intensive. Competition in the consumer market for these products exerts considerable downward pressure on pricing, and as a result, the profit margin per product is relatively low. In order to maintain or grow its market share and operate profitably, the manufacturer is required to make, package, distribute and sell the product as cost effectively and efficiently as available technology in materials and equipment, product design, and marketing and logistics practices, permit. This pressure requires constant vigilance in seeking and identifying opportunities to reduce costs while still delivering a product that pleases the market.

One product design effort has fairly recently resulted in a comparatively thinner, less bulky, and lower weight disposable diaper. For reasons of availability, cost and efficacy as liquid distribution medium, loose cellulose fiber material which may comprise comminuted wood pulp (sometimes called “airfelt”) has traditionally been used as a component of the absorbent core structure of diapers, to absorb and distribute liquid exudates discharged by the wearer. Through a change to the absorbent core structure, the recent design has a comparatively reduced need for, and therefore, quantity of, such airfelt. The design not only reduces costs through reduced usage of airfelt, but also reduces costs and usage of packaging and transportation, because more of these thinner, lighter diapers may be packaged and shipped within the same space and weight constraints. Additionally, it is believed that the thinner, less bulky design provides for greater flexibility and comfort for the wearer.

In some circumstances, a thinner design may have an effect on fit. In one respect, the greater flexibility of the thinner diaper structure, while providing greater comfort for the wearer, may in some circumstances result in a looser fit after a period of wear. This is because the diaper may flex more easily in, e.g., areas about the hips. Compensating measures, therefore, may be desired, and may add costs.

Additionally, disposable diapers are often manufactured with fastening members (sometimes called “ears”) that extend laterally from the rear waist region. These ears often bear near their distal ends a patch of hooks that constitutes one component of a hook-and-loop fastening system. The outward surface of the front waist region of the diaper may have a cooperating patch of material (often called the “landing zone”) with which the hooks are designed to engage, to effect attachment therebetween, and enable fastening of the diaper about the wearer's hips. In order to provide a stable attachment interface between the hooks and the landing zone, it may be desirable in some circumstances that the landing zone and/or hooks patch resist excessive bending, folding or wrinkling. A relatively bulky absorbent core imparts stiffness to the diaper structure that may serve this function. On the other hand, a relatively thinner, more flexible core structure may allow the diaper to bend, fold and/or wrinkle more easily. Thus, in a second respect, if excessive bending, folding and/or wrinkling is allowed to occur at particular locations of the landing zone, fastening strength, i.e., resistance to separation of fastening members from the landing zone, may be compromised. Again, compensating measures may be desired, and may add costs.

Thus, there is a need for cost-effective ways in which to preserve and/or enhance diaper fit and fastening strength while retaining the substiantial benefits of the thinner, lighter and less bulky diaper.

### BRIEF DESCRIPTION OF THE DRAWINGS

FIG. 1 is a perspective view of a disposable diaper, shown in an opened and relaxed state with wearer-facing surfaces up;

FIG. 2 is a plan view of the wearer-facing surfaces of a disposable diaper, shown stretched to its fullest dimensions against contraction induced by the presence of elastic members;

FIG. 3 is a plan view of the outward-facing surfaces of a disposable diaper, shown stretched to its fullest dimensions against contraction induced by the presence of elastic members, and illustrating the location and relative size of a Front Hip Flex Zone;

FIG. 4 is a plan view of the outward-facing surfaces of a disposable diaper, shown stretched to its fullest dimensions against contraction induced by the presence of elastic members;

FIG. 5A is a partial, schematic exploded cross-sectional view of diaper components at the location indicated at line 5-5 in FIG. 4;

FIG. 5B is an enlarged, further exploded schematic view of the portions of diaper components within the circle designated “5 B” in FIG. 5A; and

FIG. 6 is a vertical front view of components of a fixture used in the Circular Bending Stiffness Method herein.

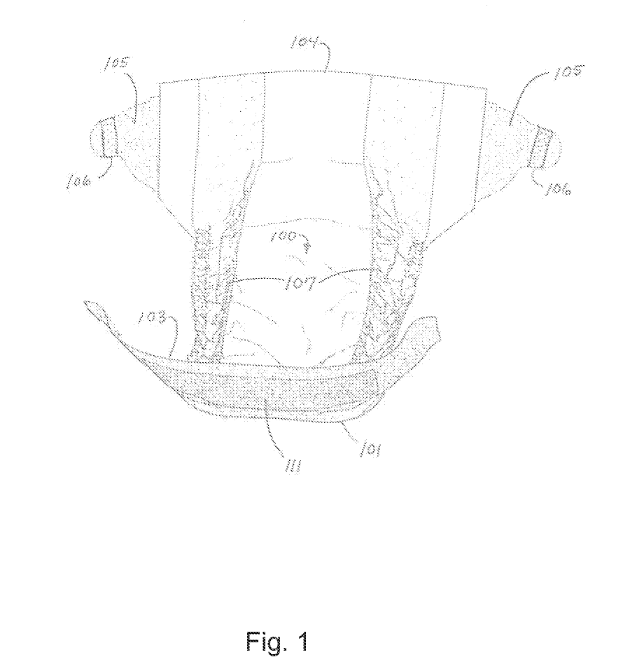

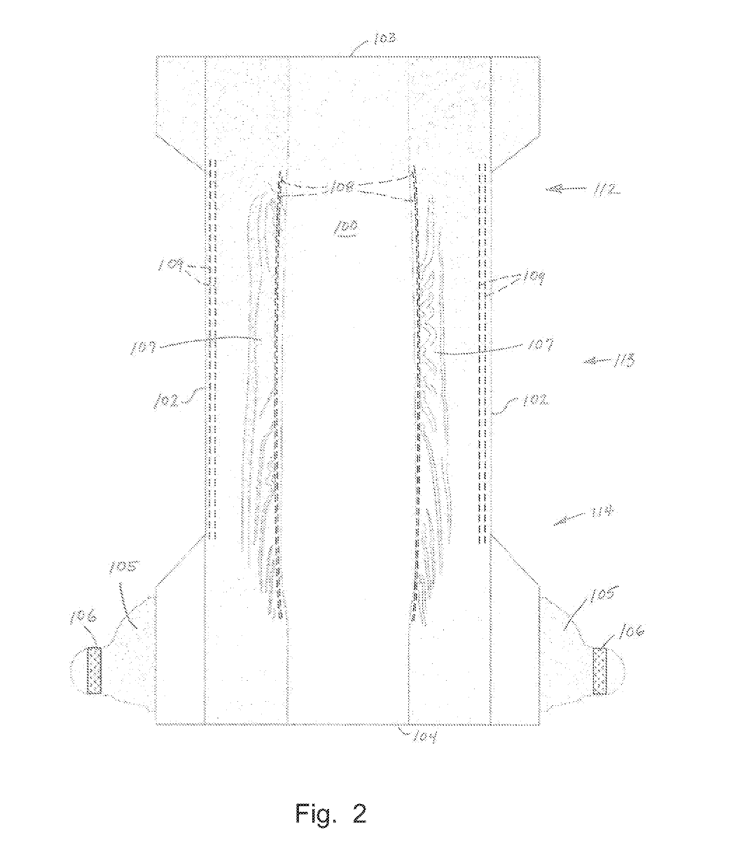

### DESCRIPTION OF EXAMPLES

Definitions

“Film” means a skin-like or membrane like layer of material formed of one or more polymers, which does not have a form consisting predominately of a web-like structure of consolidated polymer fibers and/or other fibers.

“Lateral” (and forms thereof), with respect to a disposable diaper, means along a direction extending from one side edge to the other side edge, generally parallel to the waist edges.

“Length” or a form thereof, with respect to a diaper, refers to a dimension measured along a direction gene rally perpendicular to the waist edges when the front and rear regions have been separated (such as by unfastening fastening members or severing or separating side panels) and the article, has been laid flat on a horizontal surface, and stretched out against contraction induced by elastic members.

“Longitudinal” (and forms thereof), with respect to, a disposable diaper, means along a direction extending from the front waist edge to the rear waist edge of the diaper, generally perpendicular to the waist edges.

A “nonwoven” is a manufactured sheet or web of directionally or randomly oriented fibers, consolidated and bonded together by friction, cohesion, adhesion or one or more patterns of bonds and bond impressions created through localized compression and/or application o heat or heating energy, or a combination thereof. The term does not include fabrics which are woven, knitted, or stitch-bonded with yarns or filaments. The fibers may be of natural or man-made origin and may be staple or continuous filaments or be formed in situ commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes such as meltblowing, spunbonding, solvent spinning, electrospinning, and carding. The basis weight of nonwoven fabrics is usually expressed in grams per square meter (gsm).

“Starch,” also known as amylum, means amylose and/or amylopectin in any combination, relative weight percentage, or concentration thereof.

“Width” or a form thereof, with respect to a diaper, refers to a dimension measured along a direction generally parallel to the waist edges when the front and rear regions have been separated (such as by unfastening fastening members or severing or separating side panels) and the article has been laid flat on a horizontal surface, and stretched out against contraction induced by elastic members.

“Z-direction,” with respect to a diaper, means the direction orthogonal to the length and width of the diaper as described herein.

Referring to the figures, a disposable diaper may include a central chassis having a front waist edge 103, a rear waist edge 104, and a pair of side edges 102, a front region 112, a crotch region 113 and a rear region 114. Side edges 102 may be substantially straight as shown, or may be curved laterally inwardly in some examples, for the purpose of conforming the diaper's shape about the wearer's legs through the crotch region. The front and rear regions each may include from 25 to 40 percent of the overall length L of the polymeric film component of the backsheet 101 of the diaper, and correspondingly, the crotch region 113 may include from 20 to 50 percent of the overall length of the diaper, although generally occupying its longitudinal midpoint.

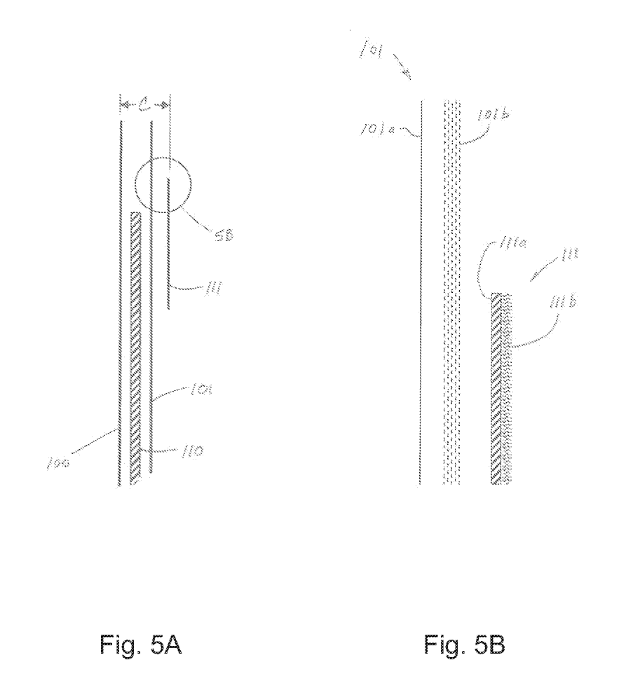

The diaper chassis may include a wearer-facing liquid permeable topsheet 100, which may be formed of a nonwoven material. Suitable examples of nonwoven topsheet materials are described in co-pending U.S. application Ser. No. 12/841,553 by Roe et at. The chassis may include an outward-facing liquid impermeable backsheet 101, which may be formed of a liquid impermeable film, or a laminate of a liquid impeimeable film and a nonwoven. For example, referring to FIG. 5B, a backsheet 101 may be formed of a laminate of a polymeric liquid impermeable film 101a and a nonwoven 101b. Film 101a and nonwoven 101b may be adhered together by a suitable adhesive. Suitable examples of backsheet materials and laminates are described further in copending U.S. application Ser. No. 13/213,198 by Kanya et al.

The chassis may also include an absorbent core 110 enveloped between the topsheet and the backsheet. Absorbent core 110 may comprise a wide variety of liquid-absorbent materials commonly used in disposable diapers and other absorbent articles such as comminuted wood pulp, which may be generally referred to as airfelt. Examples of other suitable absorbent materials include creped cellulose wadding; meltblown polymers, including coform; chemically stiffened, modified or cross-linked cellulosic fibers; tissue, including tissue wraps and tissue laminates; absorbent foams; absorbent sponges; superabsorbent polymers; absorbent gelling materials; or any other known absorbent material or combinations of materials,

Absorbent core 110 may include a liquid acquisition/distribution material layer (not specifically shown) disposed, beneath the topsheet relatively closer the wearer, and a storage material layer (not specifically shown) disposed beneath the liquid acquisition/distribution material layer. Generally, the acquisition/distribution material may have comparatively rapid absorption and wicking properties, but also may have limited absorption capacity. Conversely, generally, the storage material layer may have comparatively slower absorption and wicking properties, but also may have greater absorption capacity. Thus, the acquisition/distribution material layer may serve to rapidly absorb and distribute gushes of liquid exudate such as urine, while the storage material layer, having greater absorption capacity, may serve to absorb such liquid from the acquisition/distribution material layer and store it for the time needed until the diaper is changed.

Absorbent core 110 may be manufactured in a wide variety of sizes and shapes (e.g., rectangular, hourglass, “T”-shaped, etc.). The configuration and construction of absorbent core 110 may also be varied (e.g., the absorbent core(s) or other absorbent structure(s) may have varying caliper zones, hydrophilic gradient(s), a superabsorbent gradient(s), or lower average density and lower average basis weight acquisition zones; or may comprise one or more layers or structures). Examples of absorbent structures for use as absorbent core 110 may include those described in U.S. Pat. Nos. 4,610,678; 4,673,402: 4,834,735; 4,888,231; 5,137,537; 5,147,345; 5,342,338; 5,260,345; 5,387,207; and 5,625,222,

To reduce the overall size and/or caliper of the absorbent core, thereby reducing costs of materials and usage of packaging and shipping resources, improving wearer comfort and reducing the volume of disposable waste created by soiled diapers, it may be desired to construct an absorbent core using the lowest volumes of core materials possible within performance constraints. Toward this end, examples of suitable materials and constructions for a suitable absorbent core are described in, but are not limited to, U.S. application Ser. Nos. 12/141,122; 12/141,124; 12/141,126; 12/141,128; 12/141,130; 12/141,132; 12/141,134; 12/141,141; 12/141,143; and 12/141,146. These applications generally describe absorbent core constructions that minimize or eliminate the need for and inclusion of airfelt or other forms of cellulose fiber in combination with particles of superabsorbent polymer (hereinafter, “reduced airfelt cores”), Airfelt and other cellulose fiber have been used as absorbent fillers in absorbent cores of disposable diapers. Such fiber possesses absorbent properties and imparts some absorption capacity to an absorbent core, but also is included to provide a structural matrix to hold dispersed particles of superabsorbent polymer and/or absorbent gelling material. While inclusion of such particles enhances absorption capacity, keeping such particles suitably dispersed may be important to prevent the particles from “gel-blocking” in use as they swell with absorbed liquid, causing loss of absorption capacity. The inclusion of airfelt or other cellulose fiber as a matrix for superabsorbent particles can serve to reduce or prevent gel-blocking. However, it also imparts bulk to an absorbent core, even before absorption of any liquids.

In accordance with the disclosures in the applications identified immediately above, an, absorbent core 110 having a portion that has reduced or substantially no airfelt may be disposed between the topsheet 100 and the backsheet 101. The core 110 may include a layer formed at least in part of a substrate, having thereon distributed absorbent particles of a superabsorbent polymer or absorbent gelling material, and a thermoplastic adhesive composition capturing the distributed absorbent particles and adhering to at least portions of the substrate, thereby immobilizing the absorbent particles on or proximate to, and relative to, the substrate. The use of such a may be particularly suitable because it enables use of a relatively thinner, lighter and less bulky absorbent core, which enables construction of a relatively thinner, lighter, less bulky and more flexible diaper.

The diaper may include a pair of longitudinal harrier cuffs 107 attached proximate the side edges on the wearer-facing side. Barrier cuffs 107 may have pre-strained, longitudinally-disposed barrier cuff elastic bands or strands 108 incorporated therein. Elasticized barrier cuffs 107 will be elastically extensible and contractible along their free edges, and thus, will tend to stand up and away from the topsheet and thereby hug the wearer's body in the crotch region so as to perform a gasketing function that helps contain the wearer's exudates within the diaper as it is worn. To complement this function, additional pre-strained longitudinally-disposed leg elastic bands or strands 109 may be incorporated along the side edges 102. These may serve to stretehably gather the diaper materials about the wearer's legs, providing additional assurance of exudate containment and a neater outward appearance. Suitable non-limiting examples of such barrier cuffs and leg cuffs are described in more detail in U.S. Pat. Nos. 6,786,895; 6,420,627; 5,911,713; 5,906,603; 5,769,838; 5,624,425; 5,021,051; 4,808,178; and 4,597,760; and U.S. Application Pub. No. 2007/0239130 and U.S. application Ser. Nos. 11/195,272 and 11/158,563.

The diaper may include a pair of fastening members 105 oppositely extending laterally from the side edges 102. Fastening members 105 may be formed of a laterally elastically extensible stretch laminate material as described in, for example, co-pending U.S. application Ser. No. 12/904,212 (Kline et al.), or in U.S. Pat. Nos 5,167,897; 5,156,793; and 5,143,679; and U.S. application Ser. Nos. 10/288,095; 10/288,126; 10/429,433; 11/410,170; 11/811,130; 11/899,656; 11/899,810; 11/899/811; 11/899,812; 12/204,844; 12/204,849; 12/204,854; 12/204,858; or 12/204,864.

Fastening members 105 may have affixed thereto, near their distal ends, hooks patches 106. Hooks patches 106 may be sections of hooks material such as, for example, APLIX 963 extruded polyolefin hooks, a product of Aplix, Inc., Charlotte, N.C. Hooks patches 106 may be the hooks component of a hook-and-loop fastening system to be used by the consumer to attach the ends of the fastening members 105 to the outside of the front region of the diaper to secure it on a wearer.

Correspondingly, the outward-facing surfaces of the front region 112 may include a patch of landing zone material 111. Landing zone material 111 may be an adhered patch of loops material such as, for example, EBL Bright nonwoven loops material, a product of 3M Company, St. Paul, Minn. Alternatively, landing zone material 111 may be an adhered patch of nonwoven material that is specially adapted for use in providing fastenable engagement and suitable attachment strength when engaged with hooks. Examples of such nonwoven materials are described in, for example U.S. Pat. No. 7,789,870. In another alternative, however, some nonwoven materials used as outward-facing components of backsheet laminates may be deemed suitably lofty and to have fibers that are dense, strong enough, and sufficiently bonded within the material, to sufficiently engage with suitably designed hooks, and provide sufficient attachment strength, and thus no discrete patch of landing zone material 111 is needed. The area of the outward-facing surfaces of the front region 112 designed to provide engagement with hooks patches 106 is often called the “landing zone.” The landing zone generally lies in the front region, within 20 percent of the overall length of the diaper, from the front waist edge 103.

Within the front waist region 112 and at least partially coincident with the landing zone lies a zone herein designated the Front Hip Flex Zone 120. This zone has a length FZL and location extending along the portion of the diaper lying from 0 to about 20 percent of the overall length L of the polymeric film component of the backsheet, from the front waist edge thereof. This zone has a width Wm equal to the narrowest width of the polymeric film component of the backsheet; Wm is the width of the polymeric film component of the backsheet at its laterally narrowest point (which may be in the crotch region). The laterally outermost longitudinal boundary of the Front Hip Flex Zone 120 at each side lies along a longitudinal axis 121 tangent to the proximate side edge 102 at the laterally narrowest point of the polymeric film component of the backsheet. It is to be understood that, the Front Hip Flex Zone 120 is not a particular feature in and of itself, but rather, a designated approximate zone in the from region of any diaper in the front waist region. For purposes herein it is necessary only to locate the approximate boundaries of the Front Hip Flex Zone 120.

Without intending to be bound by theory, it is believed that stiffness (i.e. resistance to bending, folding and/or wrinkling) of the diaper structure within the Front Hip Flex Zone 120 may be important for two reasons. First, it is believed that stiffness in this Zone provides support to the diaper structure as it is worn by wearer, that helps maintain the fit of the diaper on the wearer as the wearer moves about, and/or as the diaper becomes loaded by the weight of the wearer's exudates. Thus, it is believed that if stiffness in the Front Hip Flex Zone 120 is insufficient, the diaper may be subject in some circumstances to loosened fit after a period of wear, Second, this Zone 120 may be coincident with the location at which the consumer will tend to attach the fastening members via hooks patches 106. For this reason, stiffness at particular locations within this Zone may help maintain the integrity of fastening engagement between the hooks patches and the landing zone. Conversely, if the diaper structure freely permits bending, folding and/or wrinkling in this Zone as the wearer moves about, holding strength between the hooks patches and the landing zone may be compromised unless compensating measures (e.g., selection of larger hooks patches, stiffer hooks patches, or hooks patches having more aggressive hooks structures) are employed.

When the diaper structure is relatively thicker in the Z-direction (as indicated by caliper dimension C shown in FIG. 5A) by, for example, inclusion of a substantial quantity of absorbent core material (such as airfelt), the thicker structure may help impart stiffness to the Front Hip Flex Zone. On the other hand, thinner absorbent cores such as those substantially airfelt free cores described above, while providing for a thinner, lighter, less bulky and more comfortable diaper, may impart less stiffness to the Front Flip Flex Zone 120, and thereby may in some circumstances have an effect on fit and fastener performance. Accordingly, when the diaper has a caliper C of, e.g., 3.0 mm or less in the Front Hip Flex Zone 120, it may be beneficial to enhance stiffness of the combined materials, at least in the Front Hip Flex Zone or portions thereof. Without intending to be bound by theory, it is believed desirable that the Front Hip Flex Zone 120 have a circular bending stiffness at Measurement Sites (as measured by the Circular Bending Stiffness Measurement Method described below) of at least 3.0 N, and more preferably 3.2 N and even more preferably 3.5 N for a diaper of caliper in the Front Hip Flex Zone of 3.0 mm or less, to have superior fit retention and fastening performance.

The stiffness of the combined materials in the Front Hip Flex Zone 120 may be enhanced by one or more of various measures. In one example, stiffness may be imparted to the Front Hip Flex Zone by selection of a landing zone material 111. Particular landing zone materials may have their own stiffness, and thereby may impart stiffness to the assembly of materials of which they form a component.

In another approach, any of the materials forming the backsheet and/or landing zone may be supplemented at least in the Front Hip Flex Zone or portions thereof by, e.g., addition of a supplemental layer of material such as a layer of polypropylene, polyethylene or polyester film, or application of a stiffening material in liquid or semi-liquid form that stiffens and/or hardens upon, e.g., cooling or evaporation of a solvent or medium.

In another example, a backsheet may be formed of a laminate of a polymeric film and a nonwoven. The adhesive used to adhere these components together to form the laminate imparts some stiffness to the laminate. Thus, increasing the basis weight of the adhesive, at least in the Front Hip Flex Zone or portions thereof, may impart the desired stiffness to the diaper structure. In another example, a landing zone material 111 may be adhered to the backsheet 100 by an adhesive. Similar to the preceding example, increasing the basis weight of the adhesive used, at least in the Front Hip Flex Zone or portions thereof, may contribute to imparting the desired stiffness. In another example, adhesives having differing stiffness properties may be applied at similar basis weights at locations, respectively, in areas outside the Front Hip Flex Zone, and areas within the Front Hip Flex Zone. The adhesive with the greater applied stiffness may be used within the Front Hip Flex Zone, to impart greater stiffness there.

In another example, a supplemental deposit of molten polymeric stiffening material may be applied to a surface of any of the component layers within the landing zone or Front Hip Flex Zone or portions thereof, such as a polymeric film or nonwoven layer component of the backsheet, or backing layer of a patch of landing zone material. Molten polymer may be applied, for example, by an imprinting technique such as by rollers, e.g., gravure roller.

In still another example, a deposit of a solution or dispersion, or combination thereof, of dissolved or suspended polymeric material(s) may be applied to a surface of any of the component layers within the landing zone or Front Hip Flex Zone or portions thereof. Suitable materials could include styrene homopolymers, styrene copolymers, polyvinyl acetate, polyvinyl chloride, acrylic polymers and elastomers, e.g. polychloroprene, natural rubber and nitrile rubbers. The stiffening polymeric material(s) may comprise a mixture of suitable polymeric materials. Suitable further examples include bio-sourced materials including saccharides and polysaccharides such as cellulose and starch. Upon evaporation of the solvent and/or suspension medium, the remaining deposit of polymeric material(s) may form a layer that imparts additional stiffness to the layer(s) to which they are applied.

It will be appreciated that the measures described above involve addition of supplemental materials to help impart stiffness. Thus, their use may add processing steps and material costs. Accordingly, it may be desirable to confine the use of such supplemental materials to the 112 front waist region, or more preferably, to areas within the Front Flip Flex Zone 120 or portions thereof. It may be desired that a supplemental stiffening material he added to the materials included within the Front Flip Flex Zone, but not substantially elsewhere in the diaper structure, such as, e.g., the crotch region or the rear waist region. Alternatively, the basis weight of supplemental materials may be greater or greatest in the front waist region, more preferably in an area within the Front Hip Flex Zone 120 or portions thereof, and lesser or zero outside these areas.

Stiffness-imparting measures that involve application of supplemental materials in liquid or semi-liquid form may be particularly well-suited to efficient localized application. Accordingly, it may be desired that stiffening materials in liquid or semi-liquid form be applied by use of one or a combination of gravure rolls, reverse rolls, knife-over rolls, metering rods, slot extruders, spray applicators (including pneumatic sprayers, airless sprayers, air-assisted airless sprayers, and high-volume/low-pressure sprayers), extruders, co-extruders, and air knife coaters, which may be configured and controlled to effect a defined and/or intermittent application of the stiffening material to diaper materials as they move through a manufacturing line.

With respect to the supplemental stiffening material used, as noted, adhesive material, or other polymeric material in molten or dissolved form may be employed. However, starch may be a particularly desirable alternative. Starch has advantages including relatively low cost, wide availability, ease of handling and use, non-toxicity, bio-degradability, and bio-compatibility. Accordingly, it may be desirable that stiffness be imparted to materials of the diaper be enhanced by addition of starch, preferably in front waist region 112, more preferably within the Front Hip Flex Zone 120 or portions thereof. Where starch is added, it may be desirable to dispose the starch between layers in a location where it is at least partially contained and protected, and cannot be abraded away. Thus, referring to FIG. 5B, it may be preferable that starch be applied to one of a backsheet film 101a, backsheet nonwoven 101b, or loops material backing 111a such that the starch is disposed between, e.g., backsheet film 101a and backsheet nonwoven 101b, or between backsheet nonwoven 101b and loops material backing 111a. In cases where the backsheet is formed only of film, the starch may be disposed between the backsheet film and material forming a landing zone. In another example, the starch may be applied so as to impregnate or occupy the interstitial spaces amonia fibers of the backsheet nonwoven 101b, and so lie between loops material backing 111a and backsheet film 101a.

A solution of starch, or a suspension/dispersion of starch particles, may be prepared in a suitable concentration and applied to one or more surfaces of diaper web materials by one or a combination of gravure rolls, reverse rolls, knife-over rolls, metering rods, slot coaters, spray applicators (including pneumatic sprayers, airless sprayers, air-assisted airless sprayers, and high-volume/low-pressure sprayers), extruders, co-extruders, and air knife coaters, which may be configured and controlled to effect a defined and intermittent application of the stiffening material to the diaper materials (e.g., to substantially only that portion of a material surface that will lie within the Front Hip Flex Zone or portions thereof of the finished product) as they move through a manufacturing line.

Components of the disposable diaper described herein can at least partially be comprised of bio-sourced content as described in U.S. App. Pub, No. 2007/0219521A1; U.S. App. Pub. No. 2011/0139658A1; U.S. App. Pub. No. 2011/0139657A1; U.S. App. Pub. No. 2011/0152812A1; and U.S. App. Pub. No. 2011/0139659A.1. These components include, but are not limited to, topsheet nonwovens, backsheet films, backsheet nonwovens, side panel nonwovens, barrier leg cuff nonwovens, super absorbent, nonwoven acquisition layers, core wrap nonwovens, adhesives, fastener hooks, fastener landing zone nonwovens and film bases, and supplemental stiffening materials.

In at least one embodiment, a disposable absorbent article component comprises a bio-based content value from about 10% to about 100% using ASTM D6866-10, method B, in another embodiment, from about 25% to about 75%, and in yet another embodiment, from about 50% to about 60% using ASTM D6866-10, method B.

In order to apply the methodology of ASTM D6866-10 to determine the bio-based content of any disposable absorbent article component, a representative sample of the disposable absorbent article component must be obtained for testing. In at least one embodiment, the disposable absorbent article component can be ground into particulates less than about 20 mesh using known grinding methods (e.g., Wiley® mill), and a representative sample of suitable mass taken from the randomly mixed particles.

### EXAMPLES

The caliper and circular bending stiffness in the Front Hip Flex Zone of several examples of disposable diapers manufactured by The Procter & Gamble Company were measured as described herein, All products except “PAMPERS CRUISERS (AF)” were current U.S. market CRUISERS disposable diaper products, size 4, “PAMPER CRUISERS (AF)” diapers were the CRUISERS product as sold in the U.S. prior to a change to a reduced-airfelt design. “PAMPERS CRUISERS (AFF)” are a current reduced-airfelt, thinner and lighter design.

The measurements taken averaged as follows:

| | Front | |
|---|---|---|
| | Hip Flex Zone Caliper | Front Hip Flex Zone Circular |
| | (mm) | Bending Stiffness (N) |
| PAMPERS | 4.55 | 3.393 |
| CRUISERS (AF) | | |
| PAMPERS | 4.35 | 3.641 |
| BABY DRY | | |
| LUVS | 4.78 | 3.214 |
| PAMPERS | 2.35 | 2.621 |
| CRUISERS | | |
| (AFF) | | |

Samples of PAMPERS CRUISERS (APP) product were then modified in various ways to increase circular bending stiffness in the Front Hip Flex Zone. It was found that circular bending stiffness could be increased by various measures including substituting differing patches of landing zone materials having greater bending stiffness themselves. It was found, further, that applying a deposit of starch to the hacking of the landing zone material is effective to increase circular bending stiffness. Example measurements taken of modified diapers were as follows:

| | Front Hip Flex Zone |
|---|---|
| | Circular Bending |
| Example Diaper/Modifications | Stiffness (N) |
| PAMPERS CRUISERS (AFF)/substituted | 2.93 |
| polypropylene nonwoven landing zone | |
| material, no applied starch | |
| PAMPERS CRUISERS (AFF)/substituted | 3.53 |
| polypropylene nonwoven landing zone | |
| material, starch applied to landing zone | |
| material backing at 4.1 gsm | |

It is believed that, because starch is a relatively inexpensive and easily obtainable material, application of starch to the landing, zone material or other materials forming the Front Hip Flex Zone is a cost-efficient way to impart added stiffness to the Front Hip Flex Zone.

Circular Bending Stiffness Measurement Method

Circular bending stiffness can be measured on the finished article. The measurement performed using a constant rate of extension tensile tester with computer interface (a suitable instrument is the MTS Alliance using Testworks 4 software, as available from MTS Systems Corp. Eden Prairie, Minn.) using a load cell for which the forces measured are within 10% to 90% of the limit of the cell, All linear measurements are made with a steel calibrated, ruler capable of measuring to ±1 mm and traceable to NIST. All measurement is performed in a conditioned room maintained at 23° C.±2 C° and 50%±2% relative humidity.

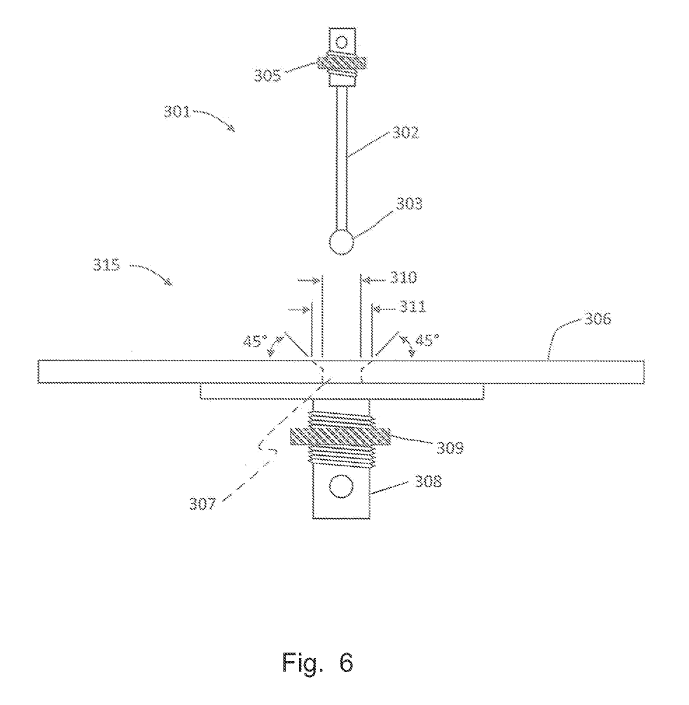

Referring to FIG. 6, the lower (stationary) fixture 315 has a horizontal plate 306 made of polished standard sheet PLEXIGLAS (Evonik Industries AG, Darmstadt, Germany) 13 mm thick by 13 cm wide by 20 cm long and a shaft 308 machined to fit the adapter to the tensile tester. The plate 306 has a circular, beveled bore 307 therethrough, centered in the XY dimension of the plate. The through-portion 310 of the bore is 18.5 mm in diameter and 13 mm deep. The upper portion of the bore is beveled at 45 degrees and has an outside diameter 311 of 29 mm. The beveled portion surface is, polished. The shaft has a locking collar 309 used to stabilize and align the plate horizontally,

The upper (movable) fixture is a plunger 301 comprising a shaft 302, 6 mm in diameter, terminated with a polished stainless steel ball 303, 11 mm in diameter. The shaft is machined to fit the adapter of the tensile tester and has a locking collar 305 used to stabilize the plunger and maintain alignment orthogonal to the plate 306 of the lower fixture and concentric to its orifice 307.

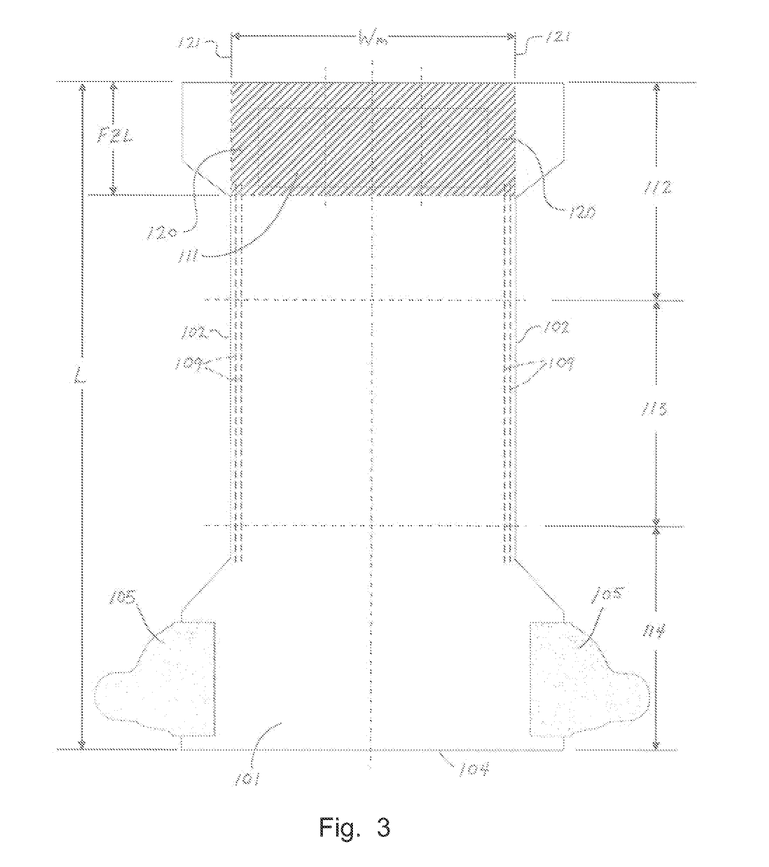

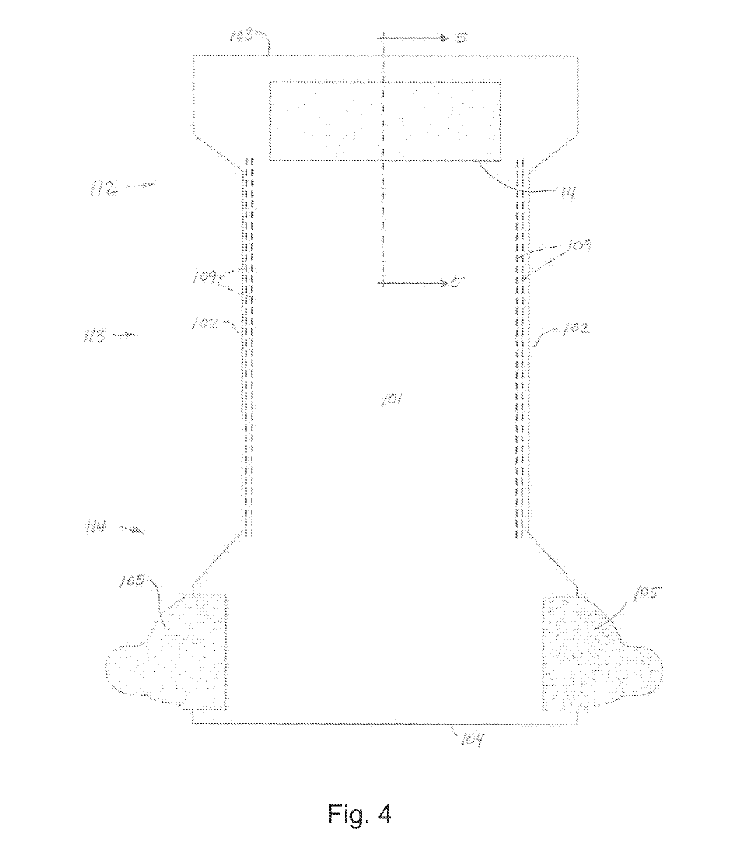

To prepare an article for measurement, condition the article at 23° C.±2 C.° and 50%±2% relative humidity for at least 2 hours prior to measurement. Open the article and place it on a lab bench with the garment-facing (outer) side upward. Locate the Front Hip Flex Zone in the front waist region, and outline its side and bottom boundaries at with a fine marker. Measure width Wm (see FIG. 3 and accompanying description above), and divide it into thirds. Using a straight edge, draw two longitudinal lines on the garment-facing side of the Front Hip Flex Zone, dividing its width Wm into three equal portions. Measure down from the front waist edge of the polymeric film component of the backsheet, a distance that is 10% of the overall length L of the polymer film component of the backsheet, and draw a lateral line along this distance which crosses the previously drawn longitudinal lines. The points at which the lateral line intersects the longitudinal lines will be the points (“Measurement Sites”) at which both caliper and circular bending stiffness are measured for purposes herein. Neatly sever the entire product along a lateral line at a distance 100 mm from the front waist edge of the polymeric film component of the backsheet; the front waist portion above the cut will be the specimen to be measured. Carefully remove or sever at close intervals any pre-strained barrier cuff elastic bands or strands, and any pm-strained leg elastic bands or strands, remaining on the specimen, to the extent necessary to keep them from preventing the specimen from laying fiat. Take care to substantially minimize any loss of absorbent core materials during cutting and subsequent handling of the specimen.

Move the cross head to 10.0 mm above the upper-surface plane of the plate 306 and zero the cross head. Program the tensile tester to perform a compression test. The crosshead is to lower at 500 mm/min for 17.0 mm, then return to its original position. Force (N) and displacement (mm) data is collected at 100 Hz.

Place the specimen, garment-facing side directed upward, on the plate 306 centering the first Measurement Site under the probe 303. Zero the crosshead and load cell, and start the test. The force at 15 mm displacement is calculated from the resulting force (N) versus displacement (mm) curve and reported to the nearest 0.01 N. Repeat measurement for the second site in like fashion. A total of 10 substantially identical samples are measured and the average of all 20 measurements reported to the nearest 0.01 N.

Caliper Measurement Method

Caliper measurements are performed using an Ono Sokki digital caliper (GS-503 Linear Gauge Sensor with DG-3610 Digital Gauge, no Sokki Co, Japan or equivalent) capable of measuring to the nearest 0.01 turn. The circular foot's diameter is 1.00 in and the applied pressure is 0.20 psi. The specimens and Measurement Sites are prepared, identified and marked in the same fashion as for the Circular Bending Stiffness Measurement Method. The caliper is first zeroed by placing the foot directly on the anvil and setting the digital gauge to zero. The foot is then raised and the specimen is placed onto the caliper anvil, with the wearer-facing surface downward and the Measurement Site centered under the foot. The foot is lowered at about 5 mm/sec until it rests on the specimen. Readings are taken after a residence time of 5 sec and recorded to the nearest 0.01 mm. The foot is raised and the measure is repeated in, like fashion at the second Measurement Site. A total of 10 substantially identical samples are measured and the average of all 20 caliper measurements reported to the nearest 0.01 mm.

Diaper Measurement Method—Locating Front Hip Flex Zone

Referring to FIG. 3, m order to measure the overall length L and narrowest width Wm of the polymer film component of a diaper backsheet for purposes of locating the Front Hip Flex Zone, stretch and flatten the diaper out by hand, against longitudinal and lateral contraction induced by any pre-strained elastic members that may be present in the bailer cuffs, leg cuffs and/or waist regions, until elastic-induced contraction is substantially eliminated, i.e., the diaper is taut along the direction of the measurement to be taken, and affix the diaper as stretched and flattened by any suitable means to a flat surface. Identify longitudinal tangent lines 121 using a straight edge, and take the measurements of the stretched diaper, as, it is affixed to the surface. Use the measurements as described above to locate the Front Hip Flex Zone. Approximate measurements for L and Wm according to this method are sufficient.

Validation of Polymers Derived from Renewable Resources A suitable validation technique is through ¹⁴C analysis. A small amount of the carbon dioxide in the atmosphere is radioactive. This ¹⁴C carbon dioxide is created when nitrogen is struck by an ultra-violet light produced neutron, causing the nitrogen to lose a proton and form carbon of molecular weight 14 which is immediately oxidized to carbon dioxide. This radioactive isotope represents a small bat measurable fraction of atmospheric carbon. Atmospheric carbon dioxide is cycled by green plants to make organic molecules during photosynthesis. The cycle is completed when the green plants or other forms of life metabolize the organic molecules, thereby producing carbon dioxide which is released back to the atmosphere. Virtually all forms of life on Earth depend on this green plant production of organic molecules to grow and reproduce. Therefore, the ¹⁴C that exists in the atmosphere becomes part of all life forms, and their biological products. En contrast, fossil fuel based carbon does not have the signature radiocarbon ratio of atmospheric carbon dioxide.

Assessment of the renewably based carbon in a material can be performed through standard test methods. Using radiocarbon and isotope ratio mass spectrometry analysis, the No-based content of materials can be determined. ASTM international, formally known as the American Society for Testing and Materials, has established a standard method for assessing the bio-based content of materials. The ASTM method is designated ASTM D6866-10,

The application of ASTM D686-10 to derive a “bio-based content” is built on the same concepts as radiocarbon dating, but without use of the age equations. The analysis is performed by deriving a ratio of the amount of organic radiocarbon (¹⁴C) in an unknown sample to that of a modern reference standard. The ratio is reported as a percentage with the units “pMC” (percent modern carbon).

The modern reference standard used in radiocarbon dating is a NIST (National Institute of Standards and Technology) standard with a known radiocarbon content equivalent approximately to the year AD 1950, AD 1950 was chosen since it represented a time prior to thermo-nuclear weapons testing which introduced large amounts of excess radiocarbon into the atmosphere with each explosion (termed “bomb carbon’). The AD 1950 reference represents 100 pMC.

“Bomb carbon” in the atmosphere reached almost twice normal levels in 1963 at the peak of testing and prior to the treaty halting the testing. Its distribution within the atmosphere has been approximated since its appearance, showing values that are greater than 100 pMC for plants and animals living since AD 1950, It has gradually decreased over time, with today's value being near 107.5 pMC. ‘This means that a fresh biomass material such as corn could give a radiocarbon signature near 107.5 pMC.

Combining fossil carbon with present day carbon into a material will result in a dilution of the present day pMC content. By presuming 107.5 pMC represents present day biomass materials and 0 pMC represents petroleum derivatives, the measured pMC value for that material will reflect the proportions of the two component types. A material derived 100% from present day soybeans would give a radiocarbon signature near 107.5 pMC. If that material was diluted with 50% petroleum derivatives, for example, it would give a radiocarbon signature near 54 pMC (assuming the petroleum derivatives have the same percentage of carbon as the soybeans).

A biomass content result is derived by assigning 100% equal to 107.5 pMC and 0% equal to 0 pMC. In this regard, a sample measuring 99 pMC will give an equivalent bio-based content value of 92%.

Assessment of the materials described herein can be done in accordance with ASTM D6866. The mean values quoted in this report encompasses an absolute range of 6% (plus and minus 3% on either side of the bio-based content value) to account for variations in end-component radiocarbon signatures. It is presumed that all materials are present day or fossil in origin and that the desired result is the amount of biobased component “present in the material, not the amount of biobased material “used” in the manufacturing process.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have, been illustrated and described, it would be obvious to those skilled in the art that various other changes and modifications can be made without departing from the spirit and scope of the invention. It is, therefore, intended to cover in the appended claims all such changes and modifications that are within the scope of this invention.

What is claimed is:

1. A disposable diaper comprising:
a chassis including a topsheet, a backsheet and an absorbent core disposed between the topsheet and the backsheet; front and rear waist edges, left and right side edges, a front region, a crotch region, and a rear region, and a Front Hip Flex Zone; and
a pair of fastening members extending laterally from the left and right side edges in the rear region, each of the fastening members having a patch of hooks material affixed thereto;
wherein a location at a Measurement Site within the Front Hip Flex Zone has a caliper no greater than 3.0 mm; and
wherein a location at a Measurement Site within the Front Hip Flex Zone has a circular bending stiffness no less than 3.0 N.

2. The diaper of claim 1 wherein the Front Flip Flex Zone comprises the topsheet, the backsheet, and at least a portion of the absorbent core.

3. The diaper of claim 2 wherein the Front Hip Flex Zone comprise a patch of landing zone material overlaying the backsheet.

4. The diaper of claim 3 wherein the patch of landing zone material is a patch of nonwoven material or a patch of loops material disposed at least partially within the Front Hip Flex Zone.

5. The diaper of claim 1 wherein the front region comprises a deposit of starch.

6. The diaper of claim 1 further comprising an overall length, and a deposit of starch lying within 20 percent of the overall length from the front waist edge.

7. The diaper of claim 1 wherein the Front Hip Flex Zone comprises a deposit of starch.

8. The diaper of claim 5 wherein the backsheet comprises a laminate of a polymeric film and a nonwoven, and the deposit of starch is disposed between the polymeric film and the nonwoven.

9. The diaper of claim 6 wherein the backsheet comprises a laminate of a polymeric film and a nonwoven, and the deposit of starch is disposed between the polymeric film and the nonwoven.

10. The diaper of claim 7 wherein the backsheet comprises a laminate of a polymeric film and a nonwoven, and the deposit of starch is disposed between the polymeric film and the nonwoven.

11. The diaper of claim 3 further comprising a deposit of starch disposed between the patch of landing zone material and the backsheet.

12. The diaper of claim 4 further comprising a deposit of starch disposed between the patch of landing zone material and the backsheet.
